(19) United States
(12) Patent Application Publication
(10) Pub. No.: US 2009/0018154 A1
(43) Pub. Date: Jan. 15, 2009

(54) OPIOID AND METHODS OF MAKING AND USING THE SAME

(75) Inventors: Huai-Hung Danny Kao, Syosset, NY (US); Yadi Zeng, Fort Lee, NJ (US); Debashis Das, Huntington Station, NY (US)

Correspondence Address: IP GROUP OF DLA PIPER US LLP, ONE LIBERTY PLACE, 1650 MARKET ST, SUITE 4900, PHILADELPHIA, PA, 19103, US

(73) Assignee: Endo Pharmaceuticals, Inc., a corporation of Delaware, Chadds Ford, PA (US)

(21) Appl. No.: 12/119,841

(22) Filed: May 13, 2008

Related U.S. Application Data
(60) Provisional application No. 60/930,615, filed on May 17, 2007.

(51) Int. Cl. A61K 31/439; C07D 489/00; A61P 29/00

(52) U.S. Cl. 514/282; 546/46

(57) ABSTRACT

An opioid derivative of oxymorphol, called 8-hydroxy-6-α-oxymorphol, has been discovered. This opioid is believed to bind at least to mu-opioid receptors and produce an analgesic or anti-tussive effect. Pharmaceutically acceptable salts of 8-hydroxy-6-α-oxymorphol, and pharmaceutical compositions comprising 8-hydroxy-6-α-oxymorphol or pharmaceutically acceptable salts thereof and pharmaceutically acceptable carrier, are also provided.

### CROSS REFERENCE TO RELATED APPLICATION

This application claims priority to U.S. Provisional Application No. 60/930,615, filed on May 17, 2007, the entire contents of which are incorporated by reference herein.

### TECHNICAL FIELD

This disclosure relates to the opioid compound 8-hydroxy-6-α-oxymorphol and its pharmaceutically acceptable salts, pharmaceutical compositions thereof, and to methods for making and using the compounds and compositions. In particular, the disclosure relates to methods for alleviating pain or cough by administering 8-hydroxy-6-α-oxymorphol and its pharmaceutically acceptable salts, or pharmaceutical compositions thereof.

### BACKGROUND

Pain is one of the conditions which is most frequently reported to health care professionals, and the treatment and management of pain is of major concern to clinicians. There are two major types of pain, nociceptive and neuropathic. Nociceptive pain results from tissue damage, and is generally responsive to analgesics such as NSAIDs and opioids. Neuropathic pain may occur when there is either damage to or dysfunction of nerves in the peripheral or central nervous system. Such nerve damage or dysfunction can be caused by trauma, burns, and external nerve compression. Opioid analgesics are also useful in treating neuropathic pain.

Coughing is a common symptom that is caused by an extremely wide range of different factors. For instance, coughing is produced by inflammatory mechanisms, mechanical disorders, and chemical and thermal stimulation of the cough receptors. Acute cough may be initially disruptive, but typically resolves within a short time and rarely requires significant medical intervention. Chronic cough can be indicative of serious respiratory diseases, and may also be the prominent symptom of certain extrapulmonary conditions (for example, upper airway and gastrointestinal disease). Even with a clear diagnosis, acute or chronic cough can be difficult to control and can be associated with impaired quality of life.

The clinical usefulness of opioids has been recognized for centuries, and morphine has long been employed as an analgesic and anti-tussive in a variety of clinical states. However, new opioids with improved pharmacokinetics and reduced side-effects are constantly being sought.

The opioid oxymorphone (14-hydroxydihydromorphinone) is indicated for the treatment of moderate to severe pain. Oxymorphone is a semisynthetic opioid agonist derived from the baine, with a significantly higher analgesic potency than that of morphine. Its structure is related to morphine, differing in a ketone group substitution at the C-6 position of morphine and saturation of the 7-8 double bond. In addition, oxymorphone has a hydroxyl group on the saturated hexane ring. The ketone group substitution makes the molecule more lipid soluble, conferring greater potency and more rapid onset of action than the hydroxylated, structurally-related compound morphine.

Oxymorphol (6-hydroxyoxymorphone) is oxymorphone which has been hydroxylated at the 6-position. This hydroxylation occurs metabolically in vivo in humans after administration of oxymorphone (see for example, Cone et al., 1983 Metabolism and Disposition vol. 11, pp 446-450). It has recently been found that oxymorphol is an active metabolite, and not only binds to opioid receptors but also causes analgesia. The 6-hydroxylation substitution of oxymorphone is made through selective reduction of the ketone group of the oxymorphone molecule, which results in a mixture of two enantiomers, 6α-oxymorphol and 6β-oxymorphol. However, these two enantiomers may have differing analgesic activities.

### SUMMARY

An opioid derivative of oxymorphol, called 8-hydroxy-6-α-oxymorphol, has been discovered. This opioid is believed to bind at least to mu-opioid receptors and produce an analgesic or anti-tussive effect. In addition to this compound, pharmaceutically acceptable salts of 8-hydroxy-6-α-oxymorphol, and pharmaceutical compositions comprising 8-hydroxy-6-α-oxymorphol (or pharmaceutically acceptable salts thereof) and pharmaceutically acceptable carrier are provided.

A method of preparing 8-hydroxy-6-α-oxymorphol is also provided. The method comprises the steps of providing 14-hydroxymorphinone and reacting this compound with sodium borohydride in an aqueous base, under conditions which allow the hydroxylation of the 8-position of 14-hydroxymorphinone prior to reduction of the ketone group.

We still further provide a method for the treatment or prevention of pain or cough in a subject, comprising administering to the subject an effective amount of 8-hydroxy-6-α-oxymorphol, a pharmaceutically acceptable salt thereof, or pharmaceutical compositions of these.

### DETAILED DESCRIPTION

The chemical structure of 8-hydroxy-6-α-oxymorphol is shown in Formula (1) below. The compound has four hydroxyl groups, which are believed to confer higher aqueous solubility than oxymorphone, oxymorphol or other opioids. The compound also has balanced hydrophilic and lipophilic properties, which are believed to allow for efficient transdermal delivery.

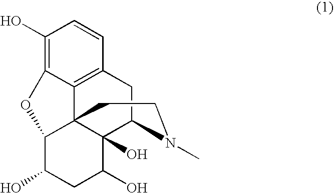

Without wishing to be bound by any theory, the physico-chemical properties of 8-hydroxy-6-α-oxymorphol, including the desirable hydrophilic-lipophilic balance, may also cause the compound to bind to opioid receptors such as the mu-, kappa- and delta-opioid receptors. In particular, 8-hydroxy-6-α-oxymorphol is believed to bind to the mu-opioid receptor.

The 8-hydroxy-6-α-oxymorphol compound can be made by reacting 14-hydroxymorphinone with sodium borohydride in an aqueous base, under conditions which allow the hydroxylation of the 8-position of 14-hydroxymorphinone prior to reduction of the ketone group. The 14-hydroxymorphinone starting material can be readily obtained by one of ordinary skill in the art. Suitable techniques for synthesizing 14-hydroxymorphinone are described in, e.g., U.S. Pat. No. 6,365,742, the entire disclosure of which is herein incorporated by reference.

The resultant 8-hydroxy-6-α-oxymorphol is a minor reaction product of the synthetic method discussed above, and can be isolated by any suitable technique known in the art. Suitable isolation techniques can comprise HPLC for example. The 8-hydroxy-6-α-oxymorphol, or a pharmaceutically acceptable salt thereof, can be further purified by recrystallization or other suitable techniques known in the art.

As used herein, “isolated” or “purified” with respect to a composition of 8-hydroxy-6-α-oxymorphol or a pharmaceutically acceptable salt thereof means that the compound is partially to substantially completely removed from the presence of other compounds. Thus, a composition comprising “isolated” or “purified” 8-hydroxy-6-α-oxymorphol can contain other chemical species, but the concentration of other chemical species is reduced as compared to the composition before it was subjected to the isolation or purification technique.

The term “pharmaceutically acceptable salt” as used herein refers to salts prepared from pharmaceutically acceptable non-toxic acids or bases, including both organic and inorganic acids and bases. Suitable pharmaceutically acceptable acid addition salts include, but are not limited to, acetic, ascorbic, benzenesulfonic (besylate), benzoic, boric, camphorsulfonic, citric, ethenesulfonic, fumaric, gluconic, glutamic, glutaric, glycerophosphoric, hydrobromic, hydrochloric, hydroiodic, isethionic, lactic, maleic, malic, malonic, mandelic, methane sulfonic, mucic, nitric, oxalic, pamoic, pantothenic, phosphoric, salicylic, succinic, sulfuric, tartaric acid, terephthalic, p-toluenesulfonic, and the like. Suitable pharmaceutically acceptable base addition salts include, but are not limited to, metallic salts made from aluminum, calcium, lithium, magnesium, potassium, sodium and zinc; organic salts made from lysine, N,N′-dibenzylethylenediamine, chloroprocaine, choline, diethanolamine, ethylenediamine, meglumine (N-methylglucamine) and procaine; and ammonium or substituted ammonium salts, for example those with lower alkylamines such as triethylamine, hydroxy alkylamines such as 2-hydroxyethylamine, bis-(2-hydroxyethyl)amine or tri-(2-hydroxyethyl)amine, cycloalkylamines such as bicyclohexylamine, dibenzylpiperidine, N-benzyl-beta-phenethylamine, dehydroabietylamine, N,N′-bisdehydroabietylamine glucamine, N-methylglucamine or bases of the pyridine type such as pyridine, collidine, quinine or quinoline.

The 8-hydroxy-6-α-oxymorphol or its pharmaceutically acceptable salts can also be formulated into pharmaceutical compositions, according to techniques known in the art. As used herein, “pharmaceutical formulations” include formulations for human and veterinary use. Pharmaceutical compositions are characterized as being suitable for administration to a human or animal subject. For example, pharmaceutical formulations for parenteral administration are desirably at least sterile and pyrogen-free. Methods for preparing pharmaceutical compositions are within the skill in the art, for example, as described in Remington's Pharmaceutical Science, 17th edit., Mack Publishing Company, Easton, Pa. (1985), the entire disclosure of which is herein incorporated by reference.

Pharmaceutical compositions can comprise from about 0.0001% to about 99% by weight of 8-hydroxy-6-α-oxymorphol or its pharmaceutically acceptable salts, for example, from about 0.01% to about 90%; from about 0.1% to about 50%, from about 1% to about 25%, or from about 5% to about 10%. Greater or lesser amounts are also contemplated.

Pharmaceutical compositions further comprise at least one pharmaceutically-acceptable carrier. The term “pharmaceutically acceptable carrier” as used herein means one or more compatible solid or liquid filler diluents, encapsulating substances or other excipients which are suitable for administration to a human or animal. The term “compatible” as used herein means that pharmaceutically acceptable carriers comprising a pharmaceutical composition are capable of being commingled with 8-hydroxy-6-α-oxymorphol or its pharmaceutically acceptable salts, and with each other, in a manner such that there is no interaction which would substantially reduce the pharmaceutical efficacy of the composition under ordinary use situations.

Suitable pharmaceutically acceptable carriers include, but are not limited to, sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as Tween; wetting agents, such as sodium lauryl sulfate; coloring agents; flavoring agents; tableting agents, stabilizers; antioxidants; preservatives; water; isotonic saline; and phosphate buffer solutions.

The choice of a pharmaceutically acceptable carrier is generally determined by the manner in which the pharmaceutical composition is to be administered. For instance, if the pharmaceutical composition is to be injected, the pharmaceutically-acceptable carrier can be sterile, physiological saline with blood-compatible suspending agent, the pH of which has been adjusted to about 7.4. Other modes of administration will are familiar to those skilled in the art, as are suitable pharmaceutically acceptable carrier(s) for each.

For example, pharmaceutically acceptable carriers suitable for the preparation of unit dosage forms for oral administration, such as tablets and capsules, are well-known in the art. Tablets and capsules typically comprise conventional pharmaceutically compatible carriers including, but not limited to, inert diluents such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of a powder mixture for formulation into a tablet or capsule. Coloring agents, such as the FD&C dyes, can be added for appearance. Sweeteners and flavoring agents, such as aspartame, saccharin, menthol, peppermint, and fruit flavors, can also be used. Capsules can also contain a liquid or gel matrix which carries the active ingredient(s).

The unit dosage forms for oral administration discussed above can also comprise extended- or controlled-release compositions known in the art. For example, the tablets or capsules discussed above can be coated by conventional methods, typically with pH- or time-dependent release coatings, such that the active agent is released over time to extend the desired action. Such dosage forms typically include, but are not limited to, coatings which comprise one or more of cellulose acetate phthalate, polyvinylacetate phthalate, hydroxypropyl methyl cellulose phthalate, ethyl cellulose, acrylic coatings, polyvinylpyrrolidone, waxes, gums and shellac. Such dosage forms can also include several times as much of active substance(s) as would be contained in an immediate-release oral pharmaceutical composition.

Oral compositions also include liquid solutions, emulsions, suspensions, and the like. The pharmaceutically acceptable carriers suitable for such compositions are well known in the art. For example, liquid oral compositions can comprise syrups, elixirs, emulsions and suspensions which include ethanol, glycerol, propylene glycol, polyethylene glycol, liquid sucrose, sorbitol and water. Typical suspending agents include, but are not limited to, methyl cellulose, sodium carboxymethyl cellulose, tragacanth and sodium alginate; typical wetting agents include, but are not limited to, lecithin and polysorbate 80; and typical preservatives include, but are not limited to, methyl paraben and sodium benzoate. Oral liquid compositions may also contain one or more components such as sweeteners, flavoring agents and colorants as discussed above.

The pharmaceutical compositions can also comprise sublingual and buccal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included. Such dosage forms may also include foaming or gas-generating agents, such as sodium carbonate and ascorbic acid.

The pharmaceutical compositions can also comprise transdermal delivery forms known in the art. For example, transdermal delivery forms typically comprise a carrier (such as, for example, a liquid, gel, or solid matrix, or a pressure sensitive adhesive) into which an active substance to be delivered is incorporated. Because the skin presents a substantial barrier to ingress of foreign substances, it is often desirable or necessary to incorporate excipients into the carrier that enhance the rate at which the active substance passes through the skin. Transdermal delivery forms known in the art include reservoir-type devices with membranes that control the rate of drug and/or skin penetration enhancer delivery to the skin. There are also “single layer” devices involving a dispersion or solution of drug and excipients in an adhesive matrix, and more complex multilaminate devices involving several distinct layers; e.g., layers for containing drug, for containing skin penetration enhancer, for controlling the rate of release of the drug and skin penetration enhancer, for attaching the device to the skin and the like.

Reservoir-type transdermal delivery forms contain a drug in a fluid or gel matrix carrier in the reservoir. In use, the drug diffuses out of the matrix and across a membrane to provide controlled release through the skin. “Single layer” transdermal delivery forms are those in which the drug is directly dispersed or dissolved in a single adhesive layer, which usually comprises a pressure sensitive adhesive matrix. Such delivery forms typically include an inert, impervious backing layer, a pressure sensitive adhesive layer containing the drug and optionally selected excipients, and a release liner that is peeled off and discarded before applying the delivery form to the skin. Examples of suitable pressure sensitive adhesives include polysiloxanes, polyacrylates, polyisobutylene, and the like. These and other transdermal delivery forms, especially those for opioids, and methods of their manufacture and use are known to those of ordinary skill in the art, for example, as described in U.S. Pat. Nos. 4,626,539; 5,762,952; 5,948,433; 5,985,317; 6,110,488; and 6,893,655, the entire disclosures of which are herein incorporated by reference.

The pharmaceutical compositions can also comprise topical delivery forms known in the art. Suitable topical delivery forms include, but are not limited to, those which comprise an admixture of 8-hydroxy-6-α-oxymorphol or its pharmaceutically acceptable salts and a skin- or mucosa-specific penetration enhancer such as lecithin. Such compositions generally contains a systemically ineffective amount of the opioid analgesic, and in any case the skin- or muscosa-specific penetration enhancer does not substantially enhance transdermal or transmucosal transmission of the opioid analgesic agent into the systemic circulation. Other suitable topical delivery forms include those which can be delivered by iontophoresis, phonophoresis and thermophoresis. Topical delivery forms suitable for delivery of opioid analgesics, and techniques for producing these, are known in the art; see, e.g., U.S. Pat. No. 6,143,278, the entire disclosure of which is herein incorporated by reference.

Pharmaceutical compositions may further comprise at least one other active pharmaceutical ingredient. Suitable additional active pharmaceutical ingredients include, but are not limited to, antihistamines, including hydroxyzine, for example, at a dosage range of from about 25 to about 400 mg; doxylamine, for example, at a dosage range of from about 3 to about 75 mg; pyrilamine, for example, at a dosage range of from about 6.25 to about 200 mg; chlorpheniramine, for example, at a dosage range of from about 1 to about 24 mg; phenindamine, for example, at a dosage range of from about 6.25 to about 150 mg; dexchlorpheniramine, for example, at a dosage range of from about 0.5 to about 12 mg; dexbrompheniramine, for example at a dosage range of from about 0.5 to about 12 mg; clemastine, for example, at a dosage range of from about 1 to about 9 mg; diphenhydramine, for example, at a dosage range of from about 6.25 to about 300 mg; azelastine, for example, at a dosage range of from about 140 to about 1,680 μg (when dosed intranasally), 1 to about 8 mg (when dosed orally); acrivastine, for example, at a dosage range of from about 1 to about 24 mg; levocarbastine (which can be dosed as an intranasal or ocular medicament), for example, at a dosage range of from about 100 to about 800 μg; mequitazine, for example, at a dosage range of from about 5 to about 20 mg; astemizole, for example, at a dosage range of from about 5 to about 20 mg; ebastine; loratadine, for example, at a dosage range of from about 5 to about 40 mg; cetirizine, for example, at a dosage range of from about 5 to about 20 mg; terfenadine, for example, at a dosage range of from about 30 to about 480 mg; terfenadine metabolites; promethazine, for example, at a dosage range of from about 6.25 to about 50 mg; dimenhydrinate, for example, at a dosage range of from about 12.5 to about 400 mg; meclizine, for example, at a dosage range of from about 6.25 to about 50 mg; tripelennamine, for example, at a dosage range of from about 6.25 to about 300 mg; carbinoxamine, for example, at a dosage range of from about 0.5 to about 16 mg; cyproheptadine, for example, at a dosage range of from about 2 to about 20 mg; azatadine, for example, at a dosage range of from about 0.25 to about 2 mg; brompheniramine, for example, at a dosage range of from about 1 to about 24 mg; triprolidine, for example, at a dosage range of from about 0.25 to about 10 mg; cyclizine, for example, at a dosage range of from about 12.5 to about 200 mg; thonzylamine, for example, at a dosage range of from about 12.5 to about 600 mg; pheniramine, for example, at a dosage range of from about 3 to about 75 mg; dextromethorphan, for example, at a dosage range of from about 2.5 to about 120 mg; noscapine, for example, at a dosage range of from about 3 to about 180 mg; benzonatate, for example, at a dosage range of from about 100 to about 600 mg; 5 diphenhydramine, for example, at a dosage range of from about 12.5 to about 150 mg; chlophedianol, for example, at a dosage range of from about 12.5 to about 100 mg; clobutinol, for example, at a dosage range of from about 20 to about 240 mg; fominoben, for example, at a dosage range of from about 80 to about 480 mg; glaucine; pholcodine, for example, at a dosage range of from about 1 to about 40 mg; zipeprol, for example, at a dosage range of from about 75 to about 300 mg; hydromorphone, for example, at a dosage range of from about 0.5 to about 8 mg; carbetapentane, for example, at a dosage range of from about 15 to about 240 mg; caramiphen, levopropoxyphene, for example, at a dosage range of from about 25 to about 200 mg and others; anti-inflammatories, for example, non-steroidal anti-inflammatories, (NSAIDS) including; ibuprofen, for example, at a dosage range of from about 50 to about 3,200 mg; naproxen, for example, at a dosage range of from about 62.5 to about 1,500 mg; sodium naproxen, for example, at a dosage range of from about 110 to about 1,650 mg; ketoprofen, for example, at a dosage range of from about 25 to about 300 mg; indoprofen, indomethacin, for example, at a dosage range of from about 25 to about 200mg; sulindac, for example, at a dosage range of from about 75 to about 400 mg; diflunisal, for example, at a dosage range of from about 125 to about 1,500 mg; ketorolac, for example, at a dosage range of from about 10 to about 120 mg; piroxicam, for example, at a dosage range of from about 10 to about 40 mg; aspirin, for example, at a dosage range of from about 80 to about 4,000 mg; meclofenamate, for example, at a dosage range of from about 25 to about 400 mg; benzydamine, for example, at a dosage range of from about 25 to about 200 mg; carprofen, for example, at a dosage range of from about 75 to about 300 mg; diclofenac, for example, at a dosage range of from about 25 to about 200 mg; etodolac, for example, at a dosage range of from about 200 to about 1,200 mg; fenbufen, for example, at a dosage range of from about 300 to about 900 mg; fenoprofen, for example, at a dosage range of from about 200 to about 3,200 mg; flurbiprofen, for example, at a dosage range of from about 50 to about 300 mg; mefenamic acid, for example, at a dosage range of from about 250 to about 1,500 mg; nabumetone, for example, at a dosage range of from about 250 to about 2,000 mg; phenylbutazone, for example, at a dosage range of from about 100 to about 400 mg; pirprofen, for example, at a dosage range of from about 100 to about 800 mg; tolmetin, for example, at a dosage range of from about 200 to about 1,800 mg and others; analgesics, including; acetaminophen, for example, at a dosage range of from about 80 to about 4,000 mg; and others: expectorants/mucolytics, including; guaifenesin, for example, at a dosage range of from about 50 to about 2,400 mg; n-acetylcysteine, for example, at a dosage range of from about 100 to about 600 mg; ambroxol, for example, at a dosage range of from about 15 to about 120 mg; bromhexine, for example, at a dosage range of from about 4 to about 64 mg; terpin hydrate, for example, at a dosage range of from about 100 to about 1,200 mg; potassium iodide, for example, at a dosage range of from about 50 to about 250 mg and others; atropinics, for example, intranasally or orally administered atropinics, including; ipratroprium (preferably intranasally), for example, at a dosage range of from about 42 to about 252 μg; atropine sulfate (preferably oral), for example, at a dosage range of from about 10 to about 1,000 μg; belladonna (for example, as an extract), for example, at a dosage range of from about 15 to about 45 mg equivalents; scopolamine, for example, at a dosage range of from about 400 to about 3,200 μg; scopolamine methobromide, for example, at a dosage range of from about 2.5 to about 20 mg; homatropine methobromide, for example, at a dosage range of from about 2.5 to about 40 mg; hyoscyamine (preferably oral), for example, at a dosage range of from about 125 to about 1,000 μg; isopropramide (preferably oral), for example, at a dosage range of from about 5 to about 20 mg; orphenadrine (preferably oral), for example, at a dosage range of from about 50 to about 400 mg; benzalkonium chloride (preferably intranasally) for example, a 0.005 to about 0.1% solution and others; mast cell stabilizers (preferably intranasally or orally administered), including; cromalyn, for example, at a dosage range of from about 10 to about 60 mg; nedocromil, for example, at a dosage range of from about 10 to about 60 mg; oxatamide, for example, at a dosage range of from about 15 to about 120 mg; ketotifen, for example, at a dosage range of from about 1 to about 4 mg; lodoxamide, for example, at a dosage range of from about 100 to about 3,000 μg and others; LT Antagonists, including zileuton and others; methylxanthines, including; caffeine, for example, at a dosage range of from about 65 to about 600 mg; theophyllene, for example, at a dosage range of from about 25 to about 1,200 mg; enprofylline; pentoxifylline, for example, at a dosage range of from about 400 to about 3,600 mg; aminophylline, for example, at a dosage range of from about 50 to about 800 mg; dyphylline, for example, at a dosage range of from about 200 to about 1,600 mg and others; antioxidants or radical inhibitors, including; ascorbic acid, for example, at a dosage range of from about 50 to about 10,000 mg; tocopherol, for example, at a dosage range of from about 50 to about 2,000 mg; ethanol, for example, at a dosage range of from about 500 to about 10,000 mg and others; steroids such as beclomethasone, for example, at a dosage range of from about 84 to about 336 μg; fluticasone, for example, at a dosage range of from about 50 to about 400 μg; budesonide, for example, at a dosage range of from about 64 to about 256 μg; mometasone; triamcinolone, for example, at a dosage range of from about 110 to about 440 μg; dexamethasone, for example, at a dosage range of from about 168 to about 1,008 μg; flunisolide, for example, at a dosage range of from about 50 to about 300 μg; prednisone (preferably oral), for example, at a dosage range of from about 5 to about 60 mg; hydrocortisone (preferably oral), for example, at a dosage range of from about 20 to about 300 mg and others; bronchodilators, for example, for inhalation, including; albuterol, for example, at a dosage range of from about 90 to about 1,080 μg; 2 to about 16 mg (if dosed orally); epinephrine, for example, at a dosage range of from about 220 to about 1,320 μg; ephedrine, for example, at a dosage range of from about 15 to about 240 mg (if dosed orally); 250 to about 1,000 μg (if dosed intranasally); metaproterenol, for example, at a dosage range of from about 65 to about 780 μg or 10 to about 80 mg if dosed orally; terbutaline, for example, at a dosage range of from about 200 to about 2,400 μg; 2.5 to about 20 mg if dosed orally; isoetharine, for example, at a dosage range of from about 340 to about 1,360 μg; pirbuterol, for example, at a dosage range of from about 200 to about 2,400 μg; bitolterol, 15 for example, at a dosage range of from about 370 to about 2,220 μg; fenoterol, for example, at a dosage range of from about 100 to about 1,200 μg; 2.5 to about 20 mg (if dosed orally); rimeterol, for example, at a dosage range of from about 200 to about 1,600 μg; ipratroprium, for example, at a dosage range of from about 18 to about 216 μg (inhalation) and others; and antivirals, including; amantadine, for example, at a dosage range of from about 50 to about 200 mg; rimantadine, for example, at a dosage range of from about 50 to about 200 mg; enviroxime; nonoxinols, for example, at a dosage range of from about 2 to about 20 mg (preferably an intranasal form); acyclovir, for example, at a dosage range of from about 200 to about 2,000 mg (oral); 1 to about 10 mg (preferably an intranasal form); alpha-interferon, for example, at a dosage range of from about 3 to about 36 MIU; beta-interferon, for example, at a dosage range of from about 3 to about 36 MIU and others; ocular drug actives: acetylcholinesterase inhibitors, e.g., echothiophate from about 0.03% to about 0.25% in topical solution and others; and gastrointestinal actives: antidiarrheals, e.g., ioperamide from about 0.1 mg to about 1.0 mg per dose, and bismuth subsalicylate from about 25 mg to about 300 mg per dose and others. Of course, clearly contemplated and included in the description above are the acid or base addition salts, esters, metabolites of these preferred actives, as well as analogues to these actives that are safe and effective. It is also recognized that an active may be useful for more than one of the above uses, and these uses are clearly contemplated as well. This overlap is recognized in the art, and adjusting dosages and the like to fit the indication is well within the purview of the skilled medical practitioner.

The pharmaceutical compositions can also comprise one or more opioids in addition to the 8-hydroxy-6-α-oxymorphol or pharmaceutical salts thereof. Suitable opioids include, but are not limited to, alfentanil, buprenorphine, butorphanol, codeine, dezocine, dihydrocodeine, fentanyl and fentanyl congeners (e.g., sufentanil, alfentanil, lofentanil, carfentanil, remifentanil, trefentanil, and mirfentanil), hydrocodone, hydromorphone, levorphanol, meperidine (pethidine), methadone, morphine, nalbuphine, oxycodone, oxymorphone, pentazocine, propiram, propoxyphene, tilidine, tramadol, the pharmaceutically acceptable acid salts thereof, and any combinations of these.

The 8-hydroxy-6-α-oxymorphol or its pharmaceutically acceptable salts, or pharmaceutical compositions thereof as discussed above can be administered to a subject in need of analgesia. As used herein, a “subject” includes a human and non-human animal. The compounds or compositions can be administered to a subject before pain is experienced (as with pain anticipated to occur after surgery), during or after the onset of pain. Such pain may be may be of any origin and any type, including nociceptive, somatic, or neuropathic. Such pain may be acute, as from an accident, or it may be chronic, as from cancer or a permanent injury. Thus, a method of treating or preventing pain by administering a therapeutically effective amount of 8-hydroxy-6-α-oxymorphol or its pharmaceutically acceptable salts, or pharmaceutical compositions thereof, is provided.

The 8-hydroxy-6-α-oxymorphol or its pharmaceutically acceptable salts, or pharmaceutical compositions thereof as discussed above can also be administered to a subject in need of an anti-tussive agent. For example, the compounds or compositions can be administered to a subject before, during or after the onset of coughing, regardless of the etiology of the cough. Thus, a method of treating or preventing cough by administering a therapeutically effective amount of 8-hydroxy-6-α-oxymorphol or its pharmaceutically acceptable salts, or pharmaceutical compositions thereof, is provided.

The therapeutically effective amount of 8-hydroxy-6-α-oxymorphol or its pharmaceutically acceptable salts, or pharmaceutical compositions thereof can be administered to a subject by any acceptable route of administration of an opioid. These routes include, but are not limited to injection (whether intrathecal, intramuscular, intravascular, intraarticular, subcutaneous or other), oral, topical, parenteral, nasal, transdermal, rectal, sublingual (including buccal) or via an implanted reservoir.

The therapeutically effective amount can be determined on an individual basis, and may be based on factors such as a subject's size, whether the subject is opioid naive, the severity of the symptoms to be treated, the duration of action desired and the result sought. In general, a therapeutically effective amount is that amount sufficient to reduce or eliminate a subject's pain or cough, but which avoids serious side effects in the subject at a reasonable benefit/risk ratio within the scope of sound medical judgment. Determination of an appropriate therapeutically effective amount of 8-hydroxy-6-α-oxymorphol or its pharmaceutically acceptable salts, or pharmaceutical compositions thereof is within the knowledge and expertise of the ordinarily skilled physician.

The actual dosage (quantity administered at a time) and the number of administrations per day comprising the therapeutically effective amount can depend on the mode of administration; for example, injection or oral administration. As with any opioid, the smallest effective dose to relieve pain should be administered as the therapeutically effective amount.

For example, a therapeutically effective amount can comprise doses from about 0.01 mg/kg to about 10 mg/kg, given intramuscularly (given, for example, every four hours). For oral administration, e.g., tablet or syrup, a therapeutically effective amount can comprise from about 0.01 mg to about 200 mg, for example, from about 0.1 mg to about 100 mg, from about 0.5 mg to about 25 mg, from about 1 mg to about 25 mg, or from about 5 mg to about 10 mg. A therapeutically effective amount in controlled (or extended) release dosage forms would be commensurately more than that of immediate release oral formulations, depending on the dosing schedule. For a twice daily dosing, a therapeutically effective amount can comprise about double to about triple the ranges above, whereas for once daily dosing, the therapeutically effective amount can comprise about four to about six times the ranges set forth above. A therapeutically effective amount in pharmaceutical compositions intended for four-hour administration can comprise from about 0.01 mg to about 100 mg per dose, for example from about 0.1 mg to about 50 mg per dose.

A variety of modifications to the aspects described will be apparent to those skilled in the art from the disclosure provided herein. Thus, the compositions and methods disclosed herein may be embodied in other specific forms without departing from the spirit or essential attributes thereof and, accordingly, reference should be made to the appended claims, rather than to the foregoing specification, as indicating the scope of our disclosure.

What is claimed:

1. 8-hydroxy-6-α-oxymorphol.

2. A composition comprising an isolated or purified 8-hydroxy-6-α-oxymorphol, or a pharmaceutically acceptable salt thereof.

3. A pharmaceutical composition comprising 8-hydroxy-6-α-oxymorphol, or a pharmaceutically acceptable salt thereof, and at least one pharmaceutically acceptable carrier.

4. The pharmaceutical composition of claim 3, comprising a dosage form selected from the group consisting of a powder, liquid, syrup, suspension, solid and semi-solid formulation.

5. The pharmaceutical composition of claim 3, comprising a topical or transdermal delivery form.

6. The pharmaceutical composition of claim 3, comprising a dosage form selected from the group consisting of a parenteral, sublingual and buccal dosage form.

7. The pharmaceutical composition of claim 3, comprising a controlled or extended release dosage form.

8. The pharmaceutical composition of claim 3, further comprising at least one active pharmaceutical ingredient other than 8-hydroxy-6-α-oxymorphol or a pharmaceutically acceptable salt thereof.

9. The pharmaceutical composition of claim 8, wherein the at least one active pharmaceutical ingredient other than 8-hydroxy-6-α-oxymorphol or a pharmaceutically acceptable salt thereof comprises an opioid.

10. A method of treating or preventing pain or cough in a subject, comprising the step of administering to the subject a therapeutically effective amount of:
(1) 8-hydroxy-6-α-oxymorphol or a pharmaceutically acceptable salt thereof; or
(2) a pharmaceutical composition comprising 8-hydroxy-6-α-oxymorphol or a pharmaceutically acceptable salt thereof and a pharmaceutically acceptable carrier.

11. The method of claim 10, wherein the 8-hydroxy-6-α-oxymorphol or pharmaceutically acceptable salt thereof, or the pharmaceutical composition, is administered by injection, orally, topically, parenterally, nasally, transdermally, rectally, sublingually or buccally, or by an implanted reservoir.

12. The method of claim 11, wherein the injection is selected from the group consisting of intrathecal, intramuscular, intravascular, intraarticular and subcutaneous injection.

13. The method of claim 10, wherein the pharmaceutical composition comprises a dosage form selected from the group consisting of a powder, liquid, syrup, suspension, solid and semi-solid formulation.

14. The method of claim 10, wherein the pharmaceutical composition comprises a topical or transdermal delivery form.

15. The method of claim 10, wherein the pharmaceutical composition comprises a dosage form selected from the group consisting of a parenteral, sublingual and buccal dosage form.

16. The method of claim 10, wherein the pharmaceutical composition further comprises at least one active pharmaceutical ingredient other than 8-hydroxy-6-α-oxymorphol or a pharmaceutically acceptable salt thereof.

17. The method of claim 16, wherein the at least one active pharmaceutical ingredient other than 8-hydroxy-6-α-oxymorphol or a pharmaceutically acceptable salt thereof comprises an opioid.
